(19) United States
(12) Patent Application Publication
(10) Pub. No.: US 2022/0279887 A1
(43) Pub. Date: Sep. 8, 2022

(54) CONCUSSION SENSING HELMET AND METHODS

(71) Applicant: Aiden J. SANDLER, Wynnewood, PA (US)

(72) Inventor: Aiden J. SANDLER, Wynnewood, PA (US)

(21) Appl. No.: 17/215,533

(22) Filed: Mar. 29, 2021

Related U.S. Application Data
(60) Provisional application No. 63/156,128, filed on Mar. 3, 2021.

(51) Int. Cl. A42B 3/04; A61B 5/00; G08B 25/10; G08B 7/06; A61B 5/0205; A61B 5/024

(52) U.S. Cl. CPC A42B 3/046; A61B 5/0022; G08B 25/10; G08B 7/06; A61B 5/7405; A61B 5/4064; A61B 5/0205; A61B 5/6803; A61B 5/742; A61B 5/024; A61B 2562/0219; A61B 2562/0247; A61B 2562/0261

(57) ABSTRACT

Concussion sensing helmet and methods of use including at least one accelerometer, at least one force sensor, at least one indicator, and a processor configured to receive and process accelerometer input from the at least one accelerometer and force sensor input from the at least one force sensor, and to instruct the at least one indicator to signal a concussion condition when the accelerometer input and the force sensor input are consistent with an impact sufficient to cause a concussion.

### BACKGROUND OF THE INVENTION

### 1. Field of Invention

This invention relates to a concussion sensing helmet and methods of using the concussion sensing helmet to determine when a concussion-triggering event has occurred.

### 2. Description of Related Art

Approximately 208 million people participate in sporting activities each year, putting these individuals at increased risk of traumatic brain injury such as concussions. A concussion occurs when a jolt or impact to the head causes the brain to move rapidly within the skull, often causing damage to brain cells or chemical changes within the brain. While testing is available to determine when a concussion has occurred, these tests frequently are not performed until days after the concussion-triggering event. The longer an individual engages in normal life with an undiagnosed concussion, the greater the chance that the individual's brain damage worsens or that the individual experiences an accidental repeat concussion.

Additionally, over-diagnosing concussions may lead to individuals being unnecessarily burdened with a concussion recovery plan, which regularly involves a temporary refrain from physical action and brain-stimulating activities such as schoolwork. In the sporting world, permitting a concussed individual to keep playing may risk long-term brain damage, but removing a non-concussed player from the game can cause an unnecessary loss of playing time. As a result, it is essential that coaching staff and on-site medical personnel be able to determine exactly when a concussion has occurred.

Prior attempts have been made to detect concussion-triggering events. For example, U.S. Pat. No. 9,026,396 B2 is directed to an impact sensing device including a plurality of accelerometers capable of producing a signal indicative of impact. The device disclosed in the '396 patent further comprises an indicator, integrated circuit, and chin guard, wherein the accelerometers and integrated circuit are incorporated within the chin guard. However, as factors such as g-force, acceleration, and stop time are all vital to determining when a specific value of acceleration or force is enough to trigger a concussion, the use of only accelerometers hinders the ability to avoid over-diagnosing or under-diagnosing concussions.

U.S. Pat. No. 8,466,794 B2 is directed to a computer-implemented method of head impact event reporting comprising collecting sensor data from sensors attached to the head of a user, determining the time passed since impact occurred, calculating an assessment score, and determining an impact vector. The system disclosed in the '794 patent further comprises sensors configured to be worn on the head of a user, a computer processor, and a memory, wherein the memory receives impact parameter information and the computer processor calculates an impact vector. However, the '794 patent does not disclose a method of precisely determining the moment when a concussion occurs, but rather generalizes when medical assistance is advised. For example, a preferred embodiment of the '794 patent discloses a “green light,” “yellow light,” and “red light” system indicating when there has been no significant impact, medical service is advised, or a player should be removed from play, respectively. Further, the '794 patent evaluates head impact as a function of passing time, rather than providing an instantaneous calculation. As a result, the '794 patent's failure to precisely calculate the moment of a concussion triggering event does not alleviate the problems of over- or under-diagnosing concussions and the significant time delay often present in the concussion evaluation process.

U.S. Pat. No. 10,338,091 B2 discloses a concussion detection and communication device comprising an electronic module being mounted to a sports helmet and a base unit configured to receive electromagnetic signals transmitted by the electronic module. The electronic module of the '091 patent comprises a controller, a transmit-receive assembly in communication with the controller, two accelerometers, and a storage unit. The base unit may comprise a personal computer, wherein the computer displays acceleration data and facilitates alerting personnel of high-impact events. While the '091 patent discloses calculating concussion from acceleration alone, the '091 patent fails to factor in pressure exerted on the head via a force sensor. As a result, the '091 patent may only achieve an approximation of whether a concussion has occurred, rather than factoring in individual-specific conditions to determine precisely when a concussion-triggering event has arisen.

Finally, U.S. Pat. Publication No. 2012/0077439 discloses a method, system, and wireless device for monitoring protective headgear. The '439 publication discloses a wireless device including a sensor module which generates data in response to impact upon the protective headgear. The sensor module comprises an accelerometer and gyroscope to calculate acceleration and rotational velocity. A short-range wireless transmitter transmits a wireless signal to a device processing module which receives the sensor data and generates concussion event data. Again, however, the '439 publication fails to factor in the g-forces, or pressure, exerted on the head, which could change the acceleration necessary to trigger a concussion causing event. As a result, while the '439 publication may approximate concussion events, it does not calculate the precise conditions necessary for a concussion to occur given various accelerations, g-forces, and stop time.

While the prior art has disclosed methods of approximating concussion-triggering events, there is a need for a device and method to detect concussion triggering events by factoring in additional variables that affect the necessary acceleration for a concussion to occur, such as pressure and stop-time. As severe head injuries may arise from both high and low speed impacts, there remains a need to factor in the specific conditions experienced by an individual to precisely determine when a concussion occurs and alert necessary personnel.

All references cited herein are incorporated herein by reference in their entireties.

### BRIEF SUMMARY OF THE INVENTION

A first aspect of the invention accordingly comprises a concussion sensing helmet, comprising: at least one accelerometer; at least one force sensor; at least one indicator; and a processor configured to receive and process accelerometer input from the at least one accelerometer and force sensor input from the at least one force sensor, and to instruct the at least one indicator to signal a concussion condition when the accelerometer input and the force sensor input are consistent with an impact sufficient to cause a concussion.

In certain embodiments, the accelerometer comprises a triple axis accelerometer.

In certain embodiments, the force sensor comprises at least one member selected from the group consisting of a strain gauge transducer, a thick film transducer, a thin film transducer, a semiconductor strain gauge transducer, a mechanical deflection sensor, a piezoelectric pressure sensor, a variable capacitance pressure instrument, and a piston-based sensor.

In certain embodiments, the processor comprises a microcontroller board.

In certain embodiments, the concussion sensing helmet further comprises a wireless communication device, wherein the wireless communication device receives input from the processor.

In certain embodiments, the wireless communication device comprises ground, VCC, RX, and TX pins.

In certain embodiments, the wireless communication device is configured to transmit data to a terminal comprising a monitor configured to display information based on the data received from the wireless communication device.

In certain embodiments, the terminal comprises a mobile electronic device.

In certain embodiments, the indicator comprises at least one member selected from the group consisting of a buzzer, a speaker, a light-emitting diode (LED) light, a liquid crystal (LCD) display, an electroluminescent (ELD) display, a plasma (PDP) display, a quantum dot (QLED) display, and a segment display.

In certain embodiments, the at least one indicator signals the concussion condition by at least one of an audio signal and a visual signal.

In certain embodiments, the processor is configured to instruct the at least one indicator to signal the concussion condition when at least one of the following is true: (a) the accelerometer input indicates acceleration in excess of a maximum level of acceleration in at least one direction; (b) the force sensor input indicates force in excess of a maximum level of force; and (c) the accelerometer input indicates a stop distance below a minimum distance.

In certain embodiments, the processor is configured to instruct the at least one indicator to signal the concussion condition when at least one of the following is true: (a) the accelerometer input indicates acceleration in excess of a hypersensitive level of acceleration lower than the maximum level of acceleration in at least one direction; (b) the force sensor input indicates force in excess of a hypersensitive level of force less than the maximum level of force; and (c) the accelerometer input indicates a stop distance less than a hypersensitive stop distance greater than the minimum distance.

In certain embodiments, the concussion sensing helmet further comprises a manually operated activator configured to enable a user to cause the at least one indicator to signal the concussion condition.

An additional aspect of the invention accordingly comprises a method for detecting when a concussion has occurred, comprising: providing the helmet of claim 1 on the head of an individual; establishing a threshold level of acceleration in at least one direction; establishing a threshold level of force; establishing a threshold stop distance; determining values of each of an acceleration, a force, and a stop distance; transmitting the values of each of the acceleration, the force, and the stop distance to a processor; and alerting others when the processor calculates values in excess of at least one of the threshold level of acceleration, the threshold level of force, and the threshold stop distance.

In certain embodiments, others are alerted via at least one of an audio alert and a visual alert.

In certain embodiments, the method further comprises transmitting input from the processor to a wireless communication device; and transmitting input from the wireless communication device to a terminal.

In certain embodiments, the method further comprises transmitting input from a terminal to a monitor; and displaying the input from the terminal on the monitor.

### BRIEF DESCRIPTION OF SEVERAL VIEWS OF THE DRAWINGS

The invention will be described in conjunction with the following drawings in which like reference numerals designate like elements and wherein:

FIG. 1 is a schematic view of an embodiment of the concussion sensing helmet of the invention.

FIG. 2 shows a schematic of an embodiment of the input transmission between the accelerometer, the force sensor, the processor, and the at least one indicator of the invention.

FIG. 3 shows a schematic of the input transmission between the accelerometer, the force sensor, the processor, the at least one indicator, the wireless communication device, the terminal, and the monitor of a preferred embodiment of the invention.

### DETAILED DESCRIPTION OF PREFERRED EMBODIMENTS OF THE INVENTION

The invention provides a system and method to sense and report when a person experiences a concussion. Preferred embodiments of the invention are particularly well suited to detect when a concussion has occurred by calculating an individual's acceleration, the force exerted upon an individual, and the individual's stop distance. Preferred embodiments of the invention alert the user and third parties as to when a concussion-triggering event has occurred.

The inventive method comprises providing the helmet of the invention on the head of an individual, establishing a threshold level of acceleration in at least one direction, establishing a threshold level of force, establishing a threshold level of stop distance, determining values of each of an acceleration, a force, and a stop distance, transmitting the values of each of the acceleration, the force, and the stop distance to a processor, and alerting others when the processor calculates values in excess of at least one of the threshold level of acceleration, the threshold level of force, and the threshold stop distance.

A concussion may be classified as a traumatic brain injury that results in an altered physical or mental state.

The concussion sensing helmet may be configured as ahead covering, including, but not limited to, a full-face helmet, modular helmet, open face helmet, bicycle helmet, riding helmet, motorcycle helmet, military helmet, or other recreational helmet.

The accelerometer is preferably a piezoelectric, piezoresistance, or capacitive accelerometer. In particularly preferred embodiments, the accelerometer is a triple axis accelerometer.

The force sensor is preferably a strain gauge transducer, a thick film transducer, a thin film transducer, a semiconductor strain gauge transducer, a mechanical deflection sensor, a piezoelectric pressure sensor, a variable capacitance pressure instrument, or a piston-based sensor. In particularly preferred embodiments, the force sensor is a pressure pad configured to measure the amount of G-forces applied to a person's head upon contact.

Preferably, the at least one indicator comprises at least one member selected from the group consisting of a buzzer, a speaker, a light-emitting diode (LED) light, a liquid crystal (LCD) display, an electroluminescent (ELD) display, a plasma (PDP) display, a quantum dot (QLED) display, and a segment display. In preferred embodiments, multiple indicators may be used. One preferred embodiment of the invention utilizes two LED light sources, an LCD display, and a buzzer indicator to provide both audio and visual alerts when a concussion-triggering event has occurred.

The processor may be classified as a device capable of receiving input and providing corresponding output. The processor preferably comprises a microcontroller board. In preferred embodiments, the processor comprises an ARDUINO UNO microcontroller board. In preferred embodiments, the processor may calculate both acceleration and stop distance from the accelerometer's input. In further embodiments, the processor receives input from the accelerometer and the force sensor and triggers the at least one indicator to signal when the input received from the accelerometer and the force sensor are consistent with a concussion-triggering event.

In preferred embodiments, the processor shall prompt the at least one indicator to signal when at least one of the following is true: (a) the accelerometer input indicates acceleration in excess of a maximum level of acceleration in at least one direction; (b) the force sensor input indicates force in excess of a maximum level of force; and (c) the accelerometer input indicates a stop distance below a minimum distance. In additional embodiments, the processor is configured to instruct the at least one indicator to signal the concussion condition when at least one of the following is true: (a) the accelerometer input indicates acceleration in excess of a hypersensitive level of acceleration lower than the maximum level of acceleration in at least one direction; (b) the force sensor input indicates force in excess of a hypersensitive level of force less than the maximum level of force; and (c) the accelerometer input indicates a stop distance less than a hypersensitive stop distance greater than the minimum distance.

In certain embodiments, the maximum level of force above which a concussion-triggering event may occur is 50, 55, 60, 65, 70, 75, 80, 85, 90, 95, 100, 105, or 110 Gs of force. In certain embodiments, the hypersensitive level of force above which a concussion-trigger event may occur is 80, 85, 90, 95, or 100 Gs of force and most preferably 90 Gs of force. In preferred embodiments, the processor may be configured to calculate the amount of force needed to cause a concussion when a user has had previous concussions. For example, in one embodiment of the invention, if a user has had one previous concussion, the processor may be configured to send input to the at least one indicator when a user experiences an impact event over 85 Gs of force instead of 90 Gs of force. In this way, the invention may accommodate individuals with previous concussion experiences, and preferred embodiments of the invention calculate a lower maximum level of force over which the processor triggers the at least one indicator for those individuals than individuals with no prior concussion history. When acceleration in excess of a maximum or hypersensitive level of acceleration has occurred is calculated based on a direct relationship calculation of the force experienced by the invention's user. When a stop distance below a minimum distance or a hypersensitive distance has occurred is calculated based on an inverse relationship with acceleration and force.

Additional embodiments of the invention may utilize a wireless communication device. The wireless communication device is configured to receive input from the processor and in preferred embodiments may comprise a BLUETOOTH module preferably comprising ground, VCC, RX, and TX pins. The wireless communication device may preferably be configured to transmit input to a terminal signaling that a concussion-triggering event has occurred when the processor receives input from the accelerometer and the force sensor that is consistent with a concussion-triggering event.

In preferred embodiments, the terminal may comprise a desktop computer or mobile electronic device, such as a laptop, cell phone, tablet, notebook, smart watch, or other similar device. In preferred embodiments, the terminal may also comprise a monitor configured to display information based on the data received from the wireless communication device. The monitor is preferably a desktop computer monitor or a screen of a mobile device, such as a laptop, cell phone, tablet, notebook, or smart watch. The terminal may further comprise the use of an application configured to display information received from the wireless communication device on the monitor.

The invention may further comprise a manually operated activator configured to enable a user to cause the at least one indicator to signal the concussion condition. In preferred embodiments, the manually operated activator is a button coupled to the user's helmet, wherein the user can push the button when medical assistance is needed. In preferred embodiments, the processor receives input from the manually operated activator. In additional preferred embodiments, the processor, upon receiving input that the manually operated activator has been activated, transmits input to the wireless communication device, wherein the wireless communication device transmits the input to a terminal comprising a monitor to alert others that the helmet-wearer needs medical assistance. In preferred embodiments, the manually operated activator, when activated, is configured to trigger the at least one indicator on the helmet of the wearer. In most preferred embodiments, the manually operated activator, when activated, will both signal audio and visual alerts via the at least one indicator on the helmet of the user and deliver input via the processor and wireless communication device to a terminal comprising a monitor to alert third parties that medical assistance is necessary.

The invention may further comprise a heart rate sensor, wherein the heart rate sensor is coupled to the helmet of the invention's user and transmits input to the processor. In some embodiments, the heart rate sensor may be coupled portion of the helmet closest to the ear lobe. When the processor calculates a heart rate in excess of a maximum level heart rate, the processor triggers the at least one indicator to signal. The processor will send input to the at least one indicator to signal when the processor calculates a heart rate below a minimum level heart rate as well. In preferred embodiments, when the processor calculates a heart rate in excess of a maximum level heart rate or below a minimum level heart rate, the processor also transmits input to the wireless communication device, which in turn transmits input to the terminal comprising a monitor to alert third parties that medical assistance is necessary.

Methods of using the invention further comprise alerting others when a concussion-triggering event has occurred via at least one of an audio alert and a visual alert. Preferred methods comprise alerting others when a concussion triggering event has occurred via both an audio alert and a visual alert.

Methods of using the invention may further comprise transmitting input from the processor to a wireless communication device; and transmitting input from the wireless communication device to a terminal. In preferred methods, the terminal further transmits input from the terminal to a monitor, wherein the monitor displays and alerts others that a concussion-triggering event has occurred.

Further methods of using the invention comprise a user activating the manually operated activator. In preferred methods, the manually operated activator is configured to enable a user to cause the at least one indicator to signal the concussion condition. In preferred methods, the manually operated activator transmits input to the processor. The processor may transmit input to at least one of the at least one indicator and the wireless communication device, wherein the wireless communication device transmits input to a terminal comprising a monitor, wherein the monitor displays a signal alerting others that the user needs medical assistance.

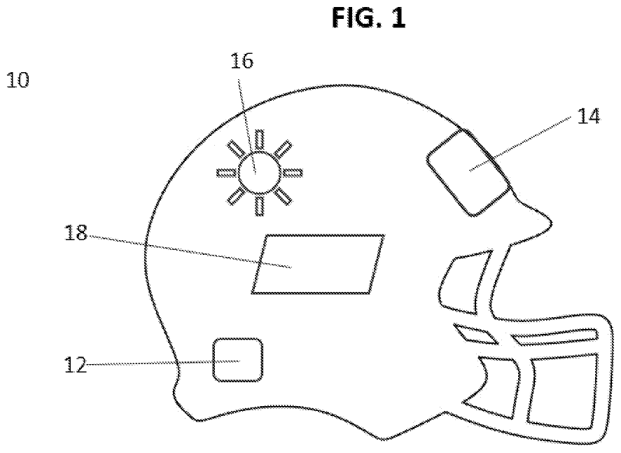

Referring to the Figures, FIG. 1 is a schematic view of an embodiment of the concussion sensing helmet 10 of the invention. The concussion sensing helmet 10 comprises an accelerometer 12, a force sensor 14, at least one indicator 16, and a processor 18. In preferred embodiments, the accelerometer 12 is a 3-plain accelerometer. In preferred embodiments, the at least one indicator 16 is configured to produce both audio and visual alerts, most preferably a buzzer sound and the illumination of LCD and LED light displays.

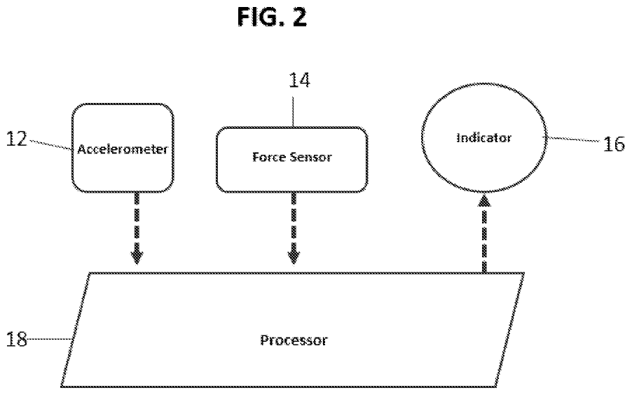

FIG. 2 shows a schematic of the input transmission between the accelerometer 12, the force sensor 14, the at least one indicator 16, and the processor 18 of an embodiment of the invention. The processor 18 receives input from the accelerometer 12 and force sensor 14 and calculates when at least one of a maximum acceleration has been exceeded, a maximum level of force has been exceeded, or a stop distance is below a minimum distance. When the processor 18 calculates at least one of the aforementioned calculations, the processor 18 transmits input to the at least one indicator 16, triggering the at least one indicator 16 to signal when a concussion-triggering event has occurred.

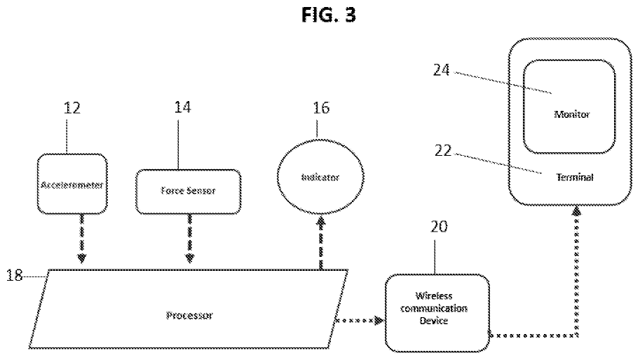

FIG. 3 shows a schematic of the input transmission between the accelerometer 12, the force sensor 14, the processor 18, the at least one indicator 16, the wireless communication device 20, the terminal 22, and the monitor 24 of a preferred embodiment of the invention. The processor 18 receives input from the accelerometer 12 and force sensor 14 and calculates when at least one of a maximum acceleration has been exceeded, a maximum level of force has been exceeded, or a stop distance is below a minimum distance. When the processor 18 calculates at least one of the aforementioned calculations, the processor 18 transmits input to the at least one indicator 16, triggering the at least one indicator 16 to signal when a concussion-triggering event has occurred. In preferred embodiments, when the processor 18 calculates at least one of the aforementioned calculations, the processor 18 additionally transmits input to a wireless communication device 20. In preferred embodiments, the wireless communication device 20 comprises a BLUETOOTH module. The wireless communication device 20 transmits input to a terminal 22 comprising a monitor 24, wherein the monitor 24 displays information received by the terminal 22.

While the invention has been described in detail and with reference to specific examples thereof, it will be apparent to one skilled in the art that various changes and modifications can be made therein without departing from the spirit and scope thereof

What is claimed is:

1. A concussion sensing helmet, comprising:
at least one accelerometer;
at least one force sensor;
at least one indicator; and
a processor configured to receive and process accelerometer input from the at least one accelerometer and force sensor input from the at least one force sensor, and to instruct the at least one indicator to signal a concussion condition when the accelerometer input and the force sensor input are consistent with an impact sufficient to cause a concussion.

2. The concussion sensing helmet of claim 1, wherein the accelerometer comprises a triple axis accelerometer.

3. The concussion sensing helmet of claim 1, wherein the force sensor comprises at least one member selected from the group consisting of a strain gauge transducer, a thick film transducer, a thin film transducer, a semiconductor strain gauge transducer, a mechanical deflection sensor, a piezoelectric pressure sensor, a variable capacitance pressure instrument, and a piston-based sensor.

4. The concussion sensing helmet of claim 1, wherein the processor comprises a microcontroller board.

5. The concussion sensing helmet of claim 1, further comprising a wireless communication device, wherein the wireless communication device receives input from the processor.

6. The concussion sensing helmet of claim 5, wherein the wireless communication device comprises ground, VCC, RX, and TX pins.

7. The concussion sensing helmet of claim 5, wherein the wireless communication device is configured to transmit data to a terminal comprising a monitor configured to display information based on the data received from the wireless communication device.

8. The concussion sensing helmet of claim 7, wherein the terminal comprises a mobile electronic device.

9. The concussion sensing helmet of claim 1, wherein the indicator comprises at least one member selected from the group consisting of a buzzer, a speaker, a light-emitting diode (LED) light, a liquid crystal (LCD) display, an electroluminescent (ELD) display, a plasma (PDP) display, a quantum dot (QLED) display, and a segment display.

10. The concussion sensing helmet of claim 1, wherein the at least one indicator signals the concussion condition by at least one of an audio signal and a visual signal.

11. The concussion sensing helmet of claim 1, wherein the processor is configured to instruct the at least one indicator to signal the concussion condition when at least one of the following is true: (a) the accelerometer input indicates acceleration in excess of a maximum level of acceleration in at least one direction; (b) the force sensor input indicates force in excess of a maximum level of force; and (c) the accelerometer input indicates a stop distance below a minimum distance.

12. The concussion sensing helmet of claim 11, wherein the processor is configured to instruct the at least one indicator to signal the concussion condition when at least one of the following is true: (a) the accelerometer input indicates acceleration in excess of a hypersensitive level of acceleration lower than the maximum level of acceleration in at least one direction; (b) the force sensor input indicates force in excess of a hypersensitive level of force less than the maximum level of force; and (c) the accelerometer input indicates a stop distance less than a hypersensitive stop distance greater than the minimum distance.

13. The concussion sensing helmet of claim 1, further comprising a manually operated activator configured to enable a user to cause the at least one indicator to signal the concussion condition.

14. The concussion sensing helmet of claim 1, further comprising a heart rate sensor, wherein the heart rate sensor is configured to deliver input to the processor.

15. A method for detecting when a concussion has occurred, comprising:
providing the helmet of claim 1 on the head of an individual;
establishing a threshold level of acceleration in at least one direction;
establishing a threshold level of force;
establishing a threshold stop distance;
determining values of each of an acceleration, a force, and a stop distance;
transmitting the values of each of the acceleration, the force, and the stop distance to a processor; and
alerting others when the processor calculates values in excess of at least one of the threshold level of acceleration, the threshold level of force, and the threshold stop distance.

16. The method of claim 15, wherein others are alerted via at least one of an audio alert and a visual alert.

17. The method of claim 15, further comprising:
transmitting input from the processor to a wireless communication device; and
transmitting input from the wireless communication device to a terminal.

18. The method of claim 17, further comprising transmitting input from a terminal to a monitor; and
displaying the input from the terminal on the monitor.
